(19) United States
(12) Patent Application Publication
(10) Pub. No.: US 2026/0118373 A1
(43) Pub. Date: Apr. 30, 2026

(54) Devices, Systems, and Methods for Chemistry Analyzer Quality Control

(71) Applicant: IDEXX LABORATORIES, Inc., Westbrook, ME (US)

(72) Inventors: Dominic Pelletier, Raymond, ME (US); Robert LaChapelle, Mechanic Falls, ME (US); Jesse Faller, Portland, ME (US); David SeeHusen, Gorham, ME (US); Zachary Helm, Kennebunk, ME (US)

(21) Appl. No.: 19/368,936

(22) Filed: Oct. 24, 2025

Related U.S. Application Data
(60) Provisional application No. 63/712,199, filed on Oct. 25, 2024.

(51) Int. Cl. G01N 33/96

(52) U.S. Cl. CPC G01N 33/96

(57) ABSTRACT

A method includes generating, from a control analyte solution, a first quality control sample of the control analyte solution. The method includes performing a first quality control test on the first quality control sample to determine a first characteristic of the first quality control sample. During the first quality control test the first quality control sample is disposed on a quality control slide. The method includes generating a first quality control response based on the first quality control test. The method includes, based on the first quality control response, generating a quality control representation. The method includes, based on the quality control representation, determining that a particular operating parameter of the chemistry analyzer is out of a predetermined tolerance. The method includes outputting an indication that the particular operating parameter is out of the predetermined tolerance.

### RELATED APPLICATION

This application claims priority to U.S. Provisional Application No. 63/712,199 filed Oct. 25, 2024 which is incorporated herein by reference in its entirety.

### FIELD OF THE DISCLOSURE

The present disclosure involves devices, systems, and methods for performing quality control on a chemistry analyzer. Namely, devices, systems, and methods of the disclosure test responses to control analyte levels at the chemistry analyzer and adjust subsystems within the chemistry analyzer based on the response.

### BACKGROUND

Chemistry analyzers can be used to calculate the concentration of certain substances, including one or more of serum, plasma, urine, or other biological fluids and/or substances.

### SUMMARY

When determining a health status of a patient (e.g., an animal patient), technicians typically use chemistry analyzers to evaluate different biological fluids and/or substances. A chemistry analyzer may use quality control fluid as a mechanism to determine specific properties (e.g., alkaline phosphatase levels, blood urea nitrogen levels, creatinine levels, glucose levels, etc.) of biological fluids and/or substances. For example, a reaction between a biological fluids and the quality control fluid may be analyzed to determine the properties of the biological fluids.

In some scenarios, quality control fluids may be lyophilized. Thus, to use the quality control fluids, the lyophilized quality control fluids have to be manually rehydrated, which may be time consuming, expensive, and prone to mistakes. These mistakes often result in low utilization and/or inaccurate quality control test responses. For example, after rehydration, the concentration of a specific control analyte in the quality control fluid may be too high or too low, which in turn, may result in inaccurate test responses, an inaccurate assessment of the chemistry analyzer's accuracy, an inaccurate assessment of the chemistry analyzer's performance, or a combination thereof.

The techniques described herein provide quality control techniques for a chemistry analyzer. In particular, the techniques described herein enable the chemistry analyzer to calibrate subsystems of the chemistry analyzer to enable the chemistry analyzer to prepare quality control fluids with accurate concentrations.

In an example embodiment, a control analyte consumable may be placed in the chemistry analyzer without customer preparation. The control analyte consumable may include (i) a first cup that includes quality control fluid having a first predetermined concentration (e.g., a high concentration) of a control analyte, (ii) a second cup that includes quality control fluid having a second predetermined concentration of the control analyte (e.g., a relatively lower concentration than the first cup), (iii) a third cup that is empty, and (iv) a quality control slide. As used herein, a “quality control slide” may correspond to a reagent slide that is usable to determine characteristics of a control analyte in a substance. For example, when the substance is applied to a quality control slide, the reaction of the quality control slide, which can be detected by one or more sensors, may be indicative of one or more different characteristics of the control analyte. In example embodiments, during downtime of the chemistry analyzer (e.g., when the chemistry analyzer is not in use by a customer or technician), automated metering capabilities of the chemistry analyzer can prepare multiple quality control samples of the quality control fluid.

For example, the chemistry analyzer can prepare quality control samples that have different known concentrations of the control analyte by performing a cross dilution process with the high concentration quality control fluid in the first cup and the relatively lower concentration quality control fluid in the second cup. In examples, the quality control slide can be used to measure the concentrations of the control analyte in the quality control samples. Based on a comparison between the measured concentrations and the known (e.g., control) concentrations, the chemistry analyzer may self-calibrate its metering capabilities-all without interrupting normal operations and/or without the user (e.g., a clinician) ever knowing.

In example embodiments, a method for performing quality control utilizing an example chemistry analyzer is disclosed. In examples, the method includes generating, from a control analyte solution, a first quality control sample of the control analyte solution. In examples, the method also includes performing a first quality control test on the first quality control sample to determine a first characteristic of the first quality control sample. In example embodiments, during the first quality control test the first quality control sample is disposed on a quality control slide. In examples, the method further includes generating a first quality control response based on the first quality control test. In examples, the method also includes, based on the first quality control response, generating a quality control representation. In example embodiments, the method further includes, based on the quality control representation, determining that a particular operating parameter of one or more components of the chemistry analyzer is out of a predetermined tolerance. In example embodiments, the method further includes outputting an indication that the particular operating parameter of the one or more components is out of the predetermined tolerance.

In another example, a chemistry analyzer is described. In some examples, the chemistry analyzer includes one or more processors and a tangible, non-transitory computer-readable medium comprising instructions that, when executed by the one or more processors, cause the one or more processors to perform operations. In examples, the operations include generating, from a control analyte solution, a first quality control sample of the control analyte solution. In examples, the operations include performing a first quality control test on the first quality control sample to determine a first characteristic of the first quality control sample. In example embodiments, during the first quality control test the first quality control sample is disposed on a quality control slide. In examples, the operations include generating a first quality control response based on the first quality control test. In examples, the operations include, based on the first quality control response, generating a quality control representation. In examples, the operations include, based on the quality control representation, determining that a particular operating parameter of one or more components of the chemistry analyzer is out of a predetermined tolerance. In examples, the operations include outputting an indication that the particular operating parameter of the one or more components is out of the predetermined tolerance.

In another example, a tangible, non-transitory computer-readable is described that includes instructions executable by one or more processors to cause one or more components (e.g., a controller of a chemistry analyzer) to perform operations. In examples, the operations include generating, from a control analyte solution, a first quality control sample of the control analyte solution. In examples, the operations include performing a first quality control test on the first quality control sample to determine a first characteristic of the first quality control sample. In example embodiments, during the first quality control test the first quality control sample is disposed on a quality control slide. In examples, the operations include generating a first quality control response based on the first quality control test. In examples, the operations include, based on the first quality control response, generating a quality control representation. In examples, the operations include, based on the quality control representation, determining that a particular operating parameter of one or more components of the chemistry analyzer is out of a predetermined tolerance. In examples, the operations include outputting an indication that the particular operating parameter of the one or more components is out of the predetermined tolerance.

In another example, a chemistry analyzer is described. In some examples, the chemistry analyzer includes a memory and a controller. In examples, the controller is configured to generate, from a control analyte solution, a first quality control sample of the control analyte solution. In examples, the controller is configured to perform a first quality control test on the first quality control sample to determine a first characteristic of the first quality control sample. In example embodiments, during the first quality control test the first quality control sample is disposed on a quality control slide. In examples, the controller is configured to generate a first quality control response based on the first quality control test. In examples, the controller is configured to, based on the first quality control response, generate a quality control representation. In examples, the controller is configured to, based on the quality control representation, determine that a particular operating parameter of one or more components of the chemistry analyzer is out of a predetermined tolerance. In examples, the controller is configured to output an indication that the particular operating parameter of the one or more components is out of the predetermined tolerance.

The features, functions, and advantages that have been discussed can be achieved independently in various examples or may be combined in yet other examples. Further details of the examples can be seen with reference to the following description and drawings.

### BRIEF DESCRIPTION OF THE FIGURES

The above, as well as additional features will be better understood through the following illustrative and non-limiting detailed description of example embodiments, with reference to the appended drawings.

FIG. 1 illustrates a block diagram of a chemistry analyzer that is configured to perform a quality control process, according to an example embodiment.

FIG. 2 illustrates a quality control process for a chemistry analyzer, according to an example embodiment.

FIG. 3 illustrates a simplified block diagram of an example computing device, according to an example embodiment.

FIG. 4 illustrates a diagram of an example computing system, according to an example embodiment.

FIG. 5 illustrates a diagram of an example computing system, according to an example embodiment.

FIG. 6 illustrates a method, according to an example embodiment.

All the figures are schematic, not necessarily to scale, and generally only show parts which are necessary to elucidate example embodiments, wherein other parts may be omitted or merely suggested.

### DETAILED DESCRIPTION

Example embodiments will now be described more fully hereinafter with reference to the accompanying drawings. That which is encompassed by the claims may, however, be embodied in many different forms and should not be construed as limited to the embodiments set forth herein; rather, these embodiments are provided by way of example. Furthermore, like numbers may refer to the same or similar elements or components throughout.

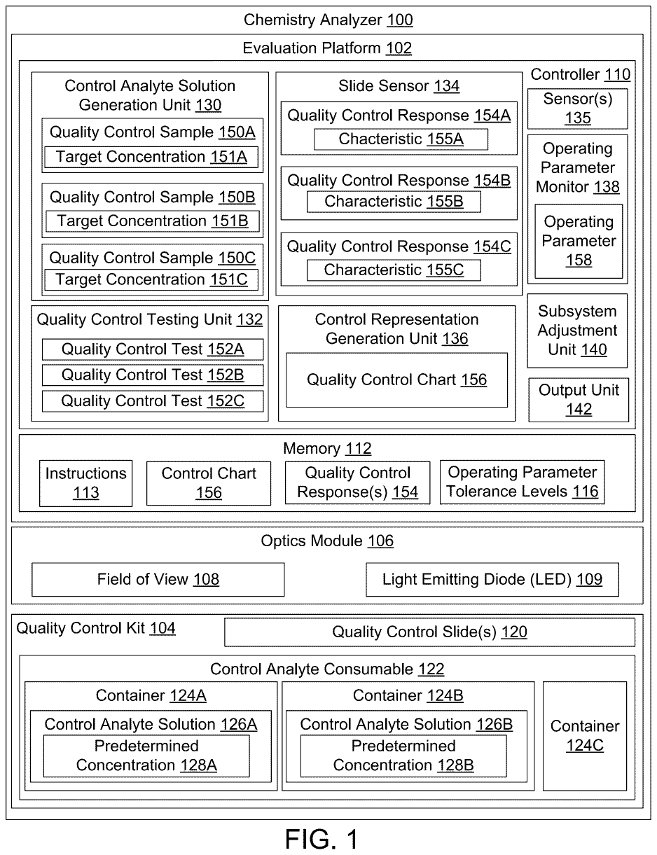

Particular implementations are described herein with reference to the figures. In the description, common features may be designated by common reference numbers throughout the drawings. In some drawings, multiple instances of a particular type of feature are used. Although these features are physically and/or logically distinct, the same reference number is used for each, and the different instances are distinguished by addition of a letter to the reference number. When the features as a group or a type are referred to herein (e.g., when no particular one of the features is being referenced), the reference number is used without a distinguishing letter. However, when one particular feature of multiple features of the same type is referred to herein, the reference number is used with the distinguishing letter. For example, referring to FIG. 1, control analyte solution is illustrated and associated with various reference numbers (e.g., 126). When referring to a particular one of the control analyte solutions, such as the control analyte solution 126A, the distinguishing letter “A” is used. However, when referring to any arbitrary one of the control analyte solutions or to the control analyte solutions as a group, one or more reference numbers (e.g., 126) may be used without a distinguishing letter.

Referring now to the figures, FIG. 1 is a block diagram of a chemistry analyzer 100 that is configured to perform a quality control process, according to an example embodiment. In particular, the chemistry analyzer 100 may be configured to utilize quality control slides to determine whether operating parameters of the chemistry analyzer 100 are calibrated to match reference operating parameters of a reference chemistry analyzer (e.g., a remote chemistry analyzer). Thus, by performing the quality control process, the chemistry analyzer 100 may determine whether subsystems within the chemistry analyzer 100 are properly operating. Furthermore, although several example components are illustrated in FIG. 1, one or more additional or alternative components may be used in connection with chemistry analyzer 100, including one or more optics modules, light emitting diodes (LEDs), one or more sensors, slide trays, cartridges, and slides (including chemistry and/or reference tile slides). The optics module and the arrangement of the slides in the chemistry analyzer 100 as described in this application may be the same as described in U.S. Pat. Nos. 7,616,317 and/or 7,588,733, the disclosures of which are incorporated herein by reference.

In examples, the chemistry analyzer 100 includes an evaluation platform 102, a quality control kit 104, and an optics module 106. In examples, the evaluation platform 102 includes a controller 110 and a memory 112. In examples, the memory 112 may be a non-transitory computer-readable medium that includes instructions 113 executable by the controller 110 to perform the operations described herein.

In examples, the controller 110 includes a control analyte solution generation unit 130, a quality control testing unit 132, a slide sensor 134, one or more additional sensors 135, a control representation generation unit 136, an operating parameter monitor 138, a subsystem adjustment unit 140 (e.g., a subsystem calibration unit), and an output unit 142. In some examples, one or more components of the controller 110 can be implemented using dedicated hardware. To illustrate, in some examples, one or more components of the controller 110 can be implemented using an application-specific integrated circuit (ASIC) or a field-programmable gate array (FPGA) device. In other examples, one or more components of the controller 110 can be implemented using software. To illustrate, in some examples, some components of the controller 110 can be implemented by executing the instructions 113 stored in the memory 112.

In examples, the quality control kit 104 includes one or more quality control slides 120 and a control analyte consumable 122. In examples, the one or more quality control slides 120 may correspond to reagent slides that are usable to determine characteristics of a control analyte in a substance. For example, when the substance is applied to a quality control slide 120, the reaction of the quality control slide 120, which can be detected by one or more sensors (e.g., the slide sensor 134 or the one or more additional sensors 135), may be indicative of one or more different characteristics of the control analyte.

In examples, the optics module 106 includes a field of view 108 and a light emitting diode (LED) 109. During operation, different quality control slides 120 can be placed in the field of view 108. In examples, when a particular quality control slide 120 is placed within the field of view 108 of the optics module 106, a quality control sample 150 is disposed on the particular quality control slide 120. The slide sensor 134 or an additional sensor 135, such as a pressure sensor, may be used to measure a characteristic 155 of the quality control sample 150 based on a response (e.g., a reaction) of the particular quality control slide 120 to the quality control sample 150. In some examples, the LED 109 may be applied to the particular quality control slide 120 to assist (e.g., aid) the sensors 134, 135 in measuring the characteristic 155.

As a non-limiting example, one or more of the sensors 134, 135 can be used to evaluate an optical response of the quality control slide 120 (e.g., a color of the quality slide, a rate at which the quality control slide changes color, a reflective density of the quality control slide, a reflectance of the quality control slide, etc.). As another non-limiting example, one or more sensors 135 may be used to evaluate a pressure response when processing or applying the quality control sample 150 to the quality control slide 120. As described in further detail below, the characteristics of the quality control slide 120 may be evaluated (e.g., compared to one or more characteristics of a reference slide and/or one or more reference operational parameters of the chemistry analyzer 100 and/or one or more chemistry analyzers that share one or more characteristics with chemistry analyzer 100) to determine whether subsystems and/or one or more components within the chemistry analyzer 100 are properly operating. In a further aspect, the above characteristics and evaluations are merely illustrative examples and should not be construed as limiting. In other embodiments, different characteristics of the quality control slide 120 may be evaluated. As a non-limiting example, an amount of time it takes to apply the quality control sample 150 to the quality control slide 120 may be reflective of a quality control condition of the chemistry analyzer 100. As another non-limiting example, a position on the quality control slide 120 in which the quality control sample 150 is applied may be reflective of a quality control condition of the chemistry analyzer 100. In some embodiments, the characteristics of the quality control slide 120 may be measured at different times to determine whether subsystems and/or one or more components within the chemistry analyzer 100 are properly operating

In some examples, once one or more substances are applied to the quality control slide 120, one or more characteristics of the quality control slide 120 may be used to determine a concentration of a control analyte in the substance (e.g., measure a control analyte solution). As non-limiting examples, the one or more quality control slides 120 can be usable to determine the concentration of alkaline phosphatase in the control analyte solution, the concentration of blood urea nitrogen in the control analyte solution, the concentration of creatinine in the control analyte solution, the concentration of glucose in the control analyte solution, etc. Thus, as a non-limiting example, the one or more quality control slides 120 can include an alkaline phosphatase slide that, when in contact with the control analyte solution, is usable to determine (e.g., calculate) the alkaline phosphatase concentration in the control analyte solution. It should be understood that the one or more quality control slides 120 may include a plurality of slides that are usable to determine the concentration of each of the above-identified control analytes. In some examples, the one or more quality control slides 120 may include a plurality of slides that are usable to determine the concentration of other control analytes.

In examples, the control analyte consumable 122 includes a container 124A, a container 124B, and a container 124C. Although three containers 124A, 124B, and 124C are depicted in FIG. 1, in other examples, the control analyte consumable 122 may include additional containers. As a non-limiting example, in some embodiments, the control analyte consumable 122 may include twenty containers. In examples, the container 124A may contain a control analyte solution 126A having a predetermined concentration 128A, the container 124B may contain a control analyte solution 126B having a predetermined concentration 128B, and the container 124C may be empty. In examples, the predetermined concentration 128B of the control analyte solution 126B may be less than the predetermined concentration 128A of the control analyte solution 126A. As a non-limiting example, if the control analyte being evaluated is glucose, the control analyte solution 126B may have a lower glucose concentration than the control analyte solution 126A. As described below, the container 124C (e.g., the empty container) may be used mix the control analyte solution 126A in the container 124A with the control analyte solution 126B in the container 124B (e.g., as part of a cross dilution process utilizing the solutions 126A and 126B).

In some examples, the control analyte solution generation unit 130 may be configured to generate the control analyte solutions 126A, 126B such that the control analyte solutions 126A, 126B are generated onboard the chemistry analyzer 100. In some examples, the control analyte solution generation unit 130 may generate the control analyte solutions 126A, 126B by rehydrating lyophilized quality control fluids. In some examples, the control analyte solution generation unit 130 may generate the control analyte solutions 126A, 126B by mixing liquid quality control fluids in containers.

During operation, the controller 110 may perform an automated quality control process to ensure that the subsystems associated with generating control analyte solutions are accurately calibrated. In examples, this quality control process may be performed during downtime of the chemistry analyzer 100 (e.g., when the chemistry analyzer 100 is not in use by a technician or customer). In examples, this quality control process may utilize one or more components of the quality control kit 104 to calibrate the subsystems. For example, the controller 110 may access the control analyte consumable 122 and the one or more quality control slides 120 to perform the automated quality control process.

To illustrate an example where the control analyte comprises glucose, one or more quality control slides 120 may correspond to a glucose slide and the predetermined concentrations 128A, 128B in the control analyte solutions 126A, 126B, respectively, may correspond to predetermined glucose concentrations. In this example embodiment, the control analyte solution generation unit 130 may be configured to generate a quality control sample 150A (of the control analyte solution 126) to have a target concentration 151A of the control analyte (e.g., glucose). In examples, to generate the quality control sample 150A, the control analyte solution generation unit 130 may be configured to perform a cross dilution process using the control analyte solutions 126A, 126B in the quality control kit 104. For example, the control analyte solution generation unit 130 may be configured to mix, in the empty container 124C, the control analyte solution 126A with the control analyte solution 126B such that the resulting mixture (e.g., the quality control sample 150A) has the target concentration 151A. Thus, in examples, the target concentration 151A of glucose may be less than the predetermined concentration 128A of glucose in the control analyte solution 126A and greater than the predetermined concentration 128B of glucose in the control analyte solution 126B. In examples, the automated metering capabilities of the control analyte solution generation unit 130 (e.g., of the chemistry analyzer 100) may be used to prepare the quality control sample 150A. For example, the automated metering capabilities of the control analyte solution generation unit 130 may be used to regulate the amounts of the control analyte solutions 126A, 126B used to generate the quality control sample 150A.

In examples, the quality control testing unit 132 may be configured to perform a quality control test 152A on the quality control sample 150A to determine a characteristic 155A of the quality control sample 150A. For example, the quality control testing unit 132 may be configured to dispose the quality control sample 150A on the glucose slide (e.g., the one or more quality control slides 120). In examples, the slide sensor 134 may be configured to generate a quality control response 154A. For example, when the quality control sample 150A is applied to the glucose slide, the slide sensor 134 may configured to determine one or more characteristics 155A of the quality control sample 150A by identifying (e.g., observing or measuring) the response of the glucose slide. The response may be an optical response, a pressure response, a positional response, etc. The slide sensor 134 may log the one or more characteristics 155A as the quality control response 154A.

The controller 110 may store the quality control response 154A in a database (e.g., the memory 112), and the control representation generation unit 136 may be configured to generate a quality control chart 156 based on the stored quality control responses 154. In examples, the quality control chart 156 may indicate the one or more characteristics 155A (e.g., the response of the glucose slide when the quality control sample 150A was applied). As described below, a difference between the one or more characteristics 155A and corresponding reference characteristics from a reference chemistry analyzer may trigger recalibration of one or more subsystems. As a non-limiting example, the metering subsystems used to generate the quality control sample 150A may be adjusted (e.g., calibrated) if the one or more characteristics 155A do not match the corresponding reference characteristics, within a degree of error.

In yet another embodiment, the chemistry analyzer 100 may perform an automated quality control process using other control analytes. To illustrate example embodiments where the control analyte comprises alkaline phosphatase, one or more quality control slides 120 may correspond to an alkaline phosphatase slide and the predetermined concentrations 128A, 128B in the control analyte solutions 126A, 126B, respectively, may correspond to predetermined alkaline phosphatase concentrations. In examples, the control analyte solution generation unit 130 may be configured to generate a quality control sample 150B (of the control analyte solution 126) to have a target concentration 151B of the control analyte (e.g., alkaline phosphatase).

In examples, to generate the quality control sample 150B, the control analyte solution generation unit 130 may be configured to perform a cross dilution process using the control analyte solutions 126A, 126B in the quality control kit 104. For example, the control analyte solution generation unit 130 may be configured to mix, in the empty container 124C, the control analyte solution 126A with the control analyte solution 126B such that the resulting mixture (e.g., the quality control sample 150B) has the target concentration 151B. Thus, in examples, the target concentration 151B of alkaline phosphatase may be less than the predetermined concentration 128A of alkaline phosphatase in the control analyte solution 126A and greater than the predetermined concentration 128B of alkaline phosphatase in the control analyte solution 126B. In examples, the automated metering capabilities of the control analyte solution generation unit 130 (e.g., of the chemistry analyzer 100) may be used to prepare the quality control sample 150B. For example, the automated metering capabilities of the control analyte solution generation unit 130 may be used to regulate the amounts of the control analyte solutions 126A, 126B used to generate the quality control sample 150B.

In examples, the quality control testing unit 132 may be configured to perform a quality control test 152B on the quality control sample 150B to determine a characteristic 155B of the quality control sample 150B. For example, the quality control testing unit 132 may be configured to dispose the quality control sample 150B on the alkaline phosphatase slide (e.g., the one or more quality control slides 120). In examples, the slide sensor 134 may be configured to generate a quality control response 154B. For example, when the quality control sample 150B is applied to the alkaline phosphatase slide, the slide sensor 134 may configured to determine one or more characteristics 155B of the quality control sample 150B by identifying (e.g., observing or measuring) the response of the alkaline phosphatase slide. The response may be an optical response, a pressure response, etc. The slide sensor 134 may log the one or more characteristics 155B as the quality control response 154B.

The controller 110 may store the quality control response 154B in the database (e.g., the memory 112), and the control representation generation unit 136 may be configured to generate (or update) the quality control chart 156 based on the stored quality control responses 154. In examples, the quality control chart 156 may indicate the one or more characteristics 155B (e.g., the response of the alkaline phosphatase slide when the quality control sample 150B was applied). As described below, a difference between the one or more characteristics 155B and corresponding reference characteristics from a reference chemistry analyzer may trigger recalibration of one or more subsystems. As a non-limiting example, the metering subsystems used to generate the quality control sample 150B may be adjusted (e.g., calibrated) if the one or more characteristics 155B do not match the corresponding reference characteristics, within a degree of error.

Similarly, to illustrate example embodiments where the control analyte is blood urea nitrogen, one or more quality control slides 120 may correspond to a blood urea nitrogen slide and the predetermined concentrations 128A, 128B in the control analyte solutions 126A, 126B, respectively, may correspond to predetermined blood urea nitrogen concentrations. In examples, the control analyte solution generation unit 130 may be configured to generate a quality control sample 150C (of the control analyte solution 126) to have a target concentration 151C of the control analyte (e.g., blood urea nitrogen). In examples, to generate the quality control sample 150C, the control analyte solution generation unit 130 may be configured to perform a cross dilution process using the control analyte solutions 126A, 126B in the quality control kit 104. For example, the control analyte solution generation unit 130 may be configured to mix, in the empty container 124C, the control analyte solution 126A with the control analyte solution 126B such that the resulting mixture (e.g., the quality control sample 150C) has the target concentration 151C. Thus, in examples, the target concentration 151C of blood urea nitrogen may be less than the predetermined concentration 128A of blood urea nitrogen in the control analyte solution 126A and greater than the predetermined concentration 128B of blood urea nitrogen in the control analyte solution 126B. In examples, the automated metering capabilities of the control analyte solution generation unit 130 (e.g., of the chemistry analyzer 100) may be used to prepare the quality control sample 150C. For example, the automated metering capabilities of the control analyte solution generation unit 130 may be used to regulate the amounts of the control analyte solutions 126A, 126B used to generate the quality control sample 150C.

In examples, the quality control testing unit 132 may be configured to perform a quality control test 152C on the quality control sample 150C to determine a characteristic 155C of the quality control sample 150C. For example, the quality control testing unit 132 may be configured to dispose the quality control sample 150C on the blood urea nitrogen slide (e.g., the one or more quality control slides 120). In examples, the slide sensor 134 may be configured to generate a quality control response 154C. For example, when the quality control sample 150C is applied to the blood urea nitrogen slide, the slide sensor 134 may configured to determine one or more characteristics 155C of the quality control sample 150C by identifying (e.g., observing or measuring) the response of the blood urea nitrogen slide. The response may be an optical response, a pressure response, etc. The slide sensor 134 may log the one or more characteristics 155C as the quality control response 154C.

The controller 110 may store the quality control response 154C in the database (e.g., the memory 112), and the control representation generation unit 136 may be configured to generate (or update) the quality control chart 156 based on the stored quality control responses 154. In examples, the quality control chart 156 may indicate the one or more characteristics 155C (e.g., the response of the blood urea nitrogen slide when the quality control sample 150C was applied). As described below, a difference between the one or more characteristics 155C and corresponding reference characteristics from a reference chemistry analyzer may trigger recalibration of one or more subsystems. As a non-limiting example, the metering subsystems used to generate the quality control sample 150C may be adjusted (e.g., calibrated) if the one or more characteristics 155C do not match the corresponding reference characteristics, within a degree of error.

It should be understood that the above processes to generate quality control responses 154 can be used for other control analytes, such as creatinine. In some embodiments, one or more of the quality control responses 154 can be determined concurrently. In some embodiments, one or more of the quality control response 154 can be determined sequentially. In some embodiments, a single quality control response 154, such as the quality control response 154A, can be determined to calibrate the subsystems.

In examples, the operating parameter monitor 138 may be configured to monitor an operating parameter 158 based on the quality control chart 156. As a non-limiting example, the operating parameter 158 may correspond to the difference between observed characteristics 155 resulting from the quality control tests 152 and the corresponding reference characteristics from the reference chemistry analyzer. In examples, based on the quality control chart 156, the operating parameter monitor 138 may be configured to determine that the operating parameter 158 is out of a predetermined tolerance (e.g., the difference exceeds a threshold). For example, the operating parameter monitor 138 may compare the operating parameter 158 to operating parameter tolerance levels 116 stored in the memory 112 to determine that the operating parameter 158 is out of the predetermined tolerance. In response to determining that the operating parameter 158 is out of the predetermined tolerance, the output unit 142 may be configured to output an indication that the particular operating parameter 158 is out of the predetermined tolerance. In some examples, the output unit 142 may display the indication to a user (e.g., a customer) via a graphical user interface.

Additionally or in the alternative, in response to determining that the operating parameter 158 is out of the predetermined tolerance, the subsystem adjustment unit 140 may be configured to adjust at least one subsystem within the chemistry analyzer 100 to bring the operating parameter 158 within the predetermined tolerance. As non-limiting examples, the subsystem adjustment unit 140 may be configured to calibrate subsystems within the control analyte solution generation unit 130 to regulate how much each control analyte solution 126A, 126B is mixed to generate the quality control samples 150.

The techniques described with respect to FIG. 1 enable the chemistry analyzer 100 to self-calibrate and prepare control analyte solutions 126 with accurate levels (e.g., concentrations). For example, based on the comparison of the characteristics 155 to reference characteristics from a reference chemistry analyzer, the chemistry analyzer 100 may adjust its automated metering parameters such that the characteristics 155 are within an acceptable margin of error. As a result, when the chemistry analyzer 100 is used by a customer or technician, the calibrated automated metering parameters of the chemistry analyzer 100 may be used to prepare quality control fluids having accurate concentrations for use in analyzing biological samples.

It should be appreciated that the quality control kit 104 may be provided in different embodiments. Thus, the configuration, properties, and materials of the quality control kit 104 depicted in FIG. 1 is not intended to be limiting. The quality control kit 104 can include liquid control fluid, dried control fluid, buffer solution, etc.

As a non-limiting example, in one embodiment, the quality control kit 104 may include one or more alkaline phosphatase slides that are lot coded such that the appropriate alkaline phosphatase concentration is calculated from the chemistry analyzer 100 response. In examples, the control analyte consumable 122 may include a sealed sample cup containing alkaline phosphatase in an appropriate buffer solution to ensure adequate long term stability when frozen such that the quality control kit 104 can be stored and transported refrigerated (or frozen). Although alkaline phosphatase is described in the above embodiment, in other embodiments, different compounds may be used, such as blood urea nitrogen, glucose, creatinine, etc.

In another embodiment, the quality control kit 104 may include one or more alkaline phosphatase slides, as described above. In this embodiment, the control analyte consumable 122 may include one sealed sample cup containing appropriate buffer solution to rehydrate dried control fluid stored in cups. In this embodiment, the control analyte consumable 122 may include one or more reagent consumable cups containing dried control fluid at one or more analyte dose levels. Although alkaline phosphatase is described in the above embodiment, in other embodiments, different compounds may be used, such as blood urea nitrogen, glucose, creatinine, etc.

In another embodiment, the quality control kit 104 may include one or more chemically inactive slides containing a spreading layer and a color registration layer to resolve color from fluid based dyes. In this embodiment, the control analyte consumable 122 may include one sealed sample cup containing dye(s) in solution.

In another embodiment, the quality control kit 104 may include one or more chemically inactive slides, as described above. In this embodiment, the control analyte consumable 122 may include one sealed sample cup containing buffer solution appropriate for dye rehydration and one or more reagent consumable cups containing dried dye solution at one or more concentration levels.

In another embodiment, the quality control kit 104 may include one or more chemically inactive slides, as described above. In this embodiment, the control analyte consumable 122 may include one sealed sample cup containing dye in solution at one or more concentration levels.

In another embodiment, the quality control kit 104 may include one or more chemically inactive slides with a central circle (or other shape) containing contrasting reference material that can be read by an optics module.

In another embodiment, the quality control kit 104 may include one or more slides with a thermally sensitive sensor material that is fluorescent or reflective colorimetric.

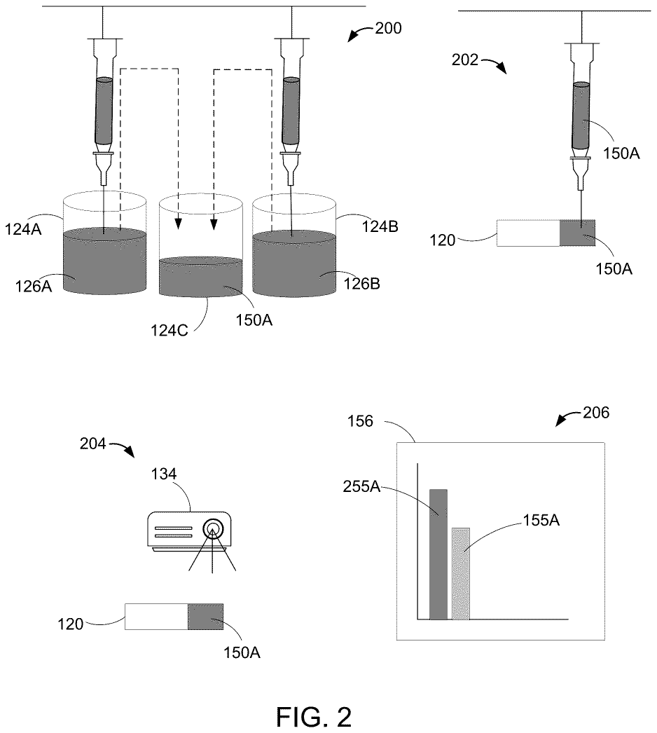

FIG. 2 illustrates a quality control process for a chemistry analyzer, according to an example embodiment. The quality control process depicted in FIG. 2 can be performed by the chemistry analyzer 100.

At process step 200, the control analyte solution generation unit 130 may be configured to generate the quality control sample 150A (of the control analyte solution 126). For example, as illustrated in FIG. 2, the control analyte solution generation unit 130 can (i) facilitate transfer of a first particular amount of the control analyte solution 126A from the container 124A to the empty container 124C and (ii) facilitate transfer of a second particular amount of the control analyte solution 126B from the container 124B to the empty container 124C. Thus, at process step 200, the control analyte solution generation unit 130 may facilitate a cross dilution process of the control analyte solution 126 to generate the quality control sample 150A. Automated metering capabilities of the control analyte solution generation unit 130 may be used to transfer specific amounts of each control analyte solution 126A, 126B such that the quality control sample 150A has the target concentration 151A of glucose. Although glucose is described, in other embodiments, different compounds may be used, such as blood urea nitrogen, glucose, creatinine, alkaline phosphatase etc.

At process step 202, the quality control testing unit 132 may be configured to perform the quality control test 152A on the quality control sample 150A to determine the characteristic 155A of the quality control sample 150A. For example, the quality control testing unit 132 may be configured to apply the quality control sample 150A to the quality control slide 120 (which contains a quality control sample of glucose). Although glucose is described, in other embodiments, different compounds may be used, such as blood urea nitrogen, glucose, creatinine, alkaline phosphatase etc.

At process step 204, the slide sensor 134 may be configured to generate the quality control response 154A. For example, when the quality control sample 150A is applied to the quality control slide 120, the slide sensor 134 may configured to identify the characteristic 155A based on the response of the quality control slide 120 (e.g., the optical response, the pressure response, etc.) to the quality control sample 150A and log the characteristic 155A as the quality control response 154A.

At process step 206, the controller 110 may generate the quality control chart 156. The quality control chart 156 may indicate the reference characteristic 255A (e.g., the reference response of the quality control slide) at the reference chemistry analyzer and the characteristic 155A (e.g., the response of the quality control slide 120) at the chemistry analyzer 100. As indicated in FIG. 2, the reference characteristic 255A and characteristic 255A may have a significant difference. In this scenario, the subsystem adjustment unit 140 may calibrate components of the control analyte solution generation unit 130 such that more of the control analyte solution 126A (e.g., the higher glucose concentration control analyte solution) and/or less control analyte solution 126B (e.g., the lower glucose concentration control analyte solution) is used when generating the quality control sample 150A. Thus, the metering parameters of the control analyte solution generation unit 130 may be calibrated. Although glucose is described, in other embodiments, different compounds may be used, such as blood urea nitrogen, glucose, creatinine, alkaline phosphatase etc.

The techniques described with respect to FIG. 2 enable the chemistry analyzer 100 to self-calibrate and prepare control analyte solutions 126 with accurate levels (e.g., concentrations). For example, based on the comparison of the characteristic 155A to reference characteristic 255A from a reference chemistry analyzer, the chemistry analyzer 100 may adjust its automated metering parameters such that the characteristics 155 are within an acceptable margin of error. As a result, when the chemistry analyzer 100 is used by a customer or technician, the calibrated automated metering parameters of the chemistry analyzer may be used to prepare quality control fluids having accurate concentrations for use in analyzing biological samples.

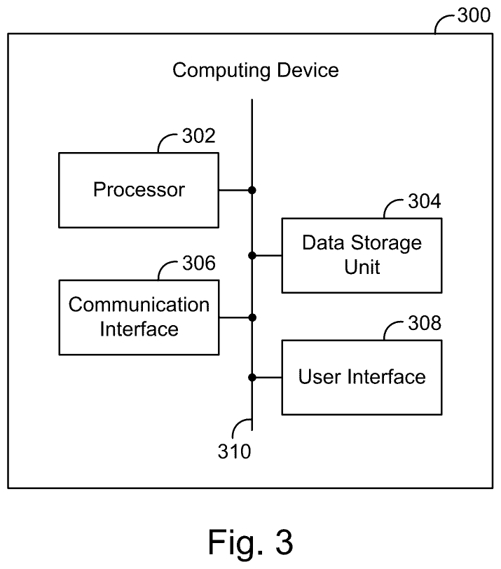

Turning to FIG. 3, FIG. 3 illustrates a simplified block diagram of an example computing device 300 of a system. In examples, the computing device 300 can correspond to the chemistry analyzer 100 of FIG. 1. In examples, the computing device 300 can include various components, such as a processor 302, a data storage unit 304, a communication interface 306, and a user interface 308. In examples, these components can be connected to each other (or to another device, system, or other entity) via connection mechanism 310.

In examples, the processor 302 can include a general-purpose processor (e.g., a microprocessor) and/or a special-purpose processor (e.g., a digital signal processor (DSP)).

In examples, the data storage unit 304 can include one or more volatile, non-volatile, removable, and/or non-removable storage components, such as magnetic, optical, or flash storage, and/or can be integrated in whole or in part with processor 302. In examples, the data storage unit 304 can take the form of a non-transitory computer-readable storage medium, having stored thereon instructions (e.g., compiled or non-compiled program logic and/or machine code) that, when executed by processor 302, cause computing device 300 to perform the operations of the controller 110.

In some instances, the computing device 300 can execute program instructions in response to receiving an input, such as from communication interface 306 and/or the user interface 308.

In examples, the communication interface 306 can allow computing device 300 to connect to and/or communicate with another other entity according to one or more protocols. In one example, the communication interface 306 can be a wired interface, such as an Ethernet interface or a high-definition serial-digital-interface (HD-SDI). In another example, the communication interface 306 can be a wireless interface, such as a cellular or WI FI interface. In this disclosure, a connection can be a direct connection or an indirect connection, the latter being a connection that passes through and/or traverses one or more entities, such as a router, switch, or other network device. Likewise, in this disclosure, a transmission can be a direct transmission or an indirect transmission.

In examples, the user interface 308 can facilitate interaction between computing device 300 and a user of computing device 300, if applicable. As such, in examples, the user interface 308 can include input components such as a keyboard, a keypad, a mouse, a touch sensitive panel, a microphone, a camera, and/or a movement sensor, all of which can be used to obtain data indicative of an environment of computing device 300, and/or output components such as a display device (which, for example, can be combined with a touch sensitive panel), a sound speaker, and/or a haptic feedback system. More generally, in examples, the user interface 308 can include hardware and/or software components that facilitate interaction between computing device 300 and the user of the computing device 300. Other examples are possible.

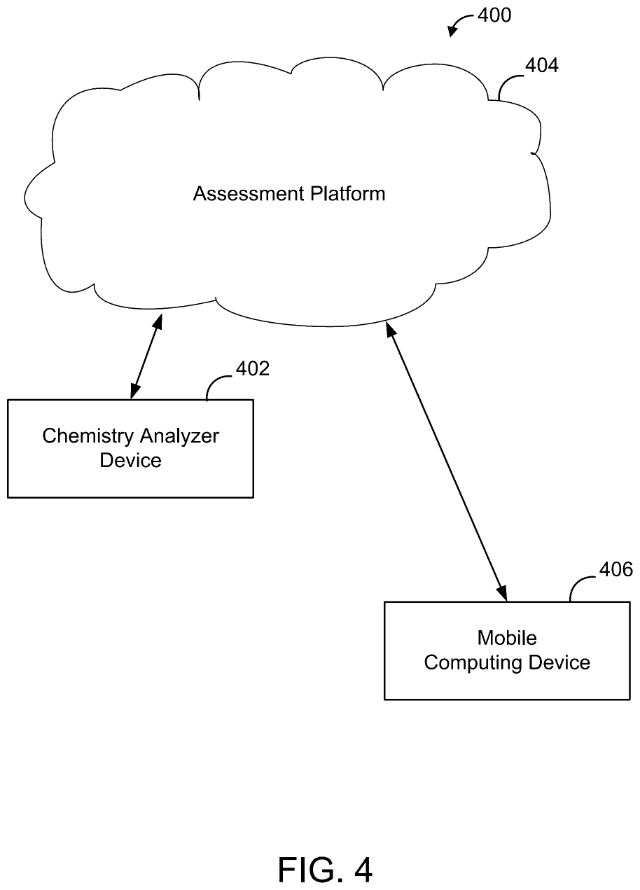

Now referring to FIG. 4, a computing system 400 configured for use with a chemistry analyzer device 402 and a mobile computing device 406 is illustrated, according to an example embodiment.

In examples, the chemistry analyzer device 402 includes a computing device, such as the computing device illustrated in FIG. 1 and/or computing device 300. It should also be readily understood that computing device 300 and the chemistry analyzer device 402, and all of the components thereof, can be physical systems made up of physical devices, cloud-based systems made up of cloud-based devices that store program logic and/or data of cloud-based applications and/or services (e.g., perform at least one function of a software application or an application platform for computing systems and devices detailed herein), or some combination of the two.

In any event, the computing system 400 can include various components, such as the computing device 300, chemistry analyzer device 402, and a cloud-based assessment platform 404.

The chemistry analyzer device 402 and/or components thereof can perform various acts and/or functions (many of which are described above). Examples of these and related features will now be described in further detail.

The chemistry analyzer device 402 may collect data from a number of sources. In one example, the chemistry analyzer device 402 may collect data from a database of values (e.g., quality control responses and/or concentration values), parameters, and/or images related to the calibration of chemistry analyzer device 402 and/or the testing of biological samples therein. These values (e.g., quality control responses and/or concentration values), parameters, and/or images may be uploaded to an assessment platform 404 and characteristics of these values, parameters, and/or images may be output to a mobile computing device 406.

In an example, the assessment platform 404 may collect data from one or more sensors communicably coupled to the chemistry analyzer device 402 (such as the sensor 134), concerning a particular substance and/or slide. In such examples, the assessment platform 404 may identify a characteristic of the slide or a testing response and transmit instructions to the mobile computing device 406 to cause a graphical user interface to display a graphical indication of the identified characteristic and/or testing response. In some examples, the assessment platform 404 may analyze and evaluate a calibration parameter and/or testing response by utilizing one or more of: (i) an artificial neural network, (ii) a support vector machine, (iii) a regression tree, or (iv) an ensemble of regression trees.

In some examples, values (e.g., quality control responses 154), parameters, and/or images that are captured by the chemistry analyzer device 402 can be stored within a memory, such as a memory of chemistry analyzer 100 and/or computing device 300, to be subsequently analyzed.

For example, in some embodiments, the chemistry analyzer device 402 may generate, from a control analyte solution having a predetermined concentration, a quality control sample for the control analyte solution. The chemistry analyzer device 402 may perform a quality control test on the quality control sample to determine a characteristic of the quality control sample. Based on the quality control test, the chemistry analyzer device 402 may generate a quality control response (e.g., indicating whether characteristics differs from a reference characteristic generated at a reference chemistry analyzer). The chemistry analyzer device 402 may store the quality control response and may generate a quality control chart based on the quality control response. Based on the quality control chart, the chemistry analyzer device 402 may determine whether a particular operating parameter (e.g., metering parameter used to generate the quality control sample) is out of the predetermined tolerance and may adjust an internal subsystem to bring the operating parameter within the predetermined tolerance. Additionally or alternatively, based on the quality control chart, a customer may be notified if the measured or observed characteristics differ from the reference characteristics generated at a reference chemistry analyzer. Additionally or alternatively, in examples, chemistry analyzer device 402 may transmit one or more of these values to assessment platform 404 for further analysis, including for updated values and/or instructions determined for chemistry analyzer device 402 by assessment platform 404 based on the transmitted data.

In one example, the assessment platform 404 may train a machine learning model using data associated values (e.g., reflectance values), parameters, and/or images of one or more slides that share a characteristic with previously captured values (e.g., reflectance values), parameters, and/or images of one or more slides. The machine learning model may be trained using training data that shares a characteristic and/or testing response with the slides (and the substances thereon and/or characteristics thereof) to be further analyzed by the chemistry analyzer device 402, assessment platform 404, or both. Training the machine learning model may include inputting one or more training values, parameters, and/or images into the machine learning model, predicting, by the machine learning model, an outcome of a determined condition of the one or more training values, parameters, and/or images, comparing the at least one outcome to the characteristic of the one or more training values, parameters, and/or images, and adjusting, based on the comparison, the machine learning model.

In some examples, the training data may include labeled input values, parameters, and/or images (supervised learning), partially labeled input values, parameters, and/or images (semi-supervised learning), or unlabeled input values, parameters, and/or images (unsupervised learning). In some examples, training may include reinforcement learning.

The machine learning model may include an artificial neural network, a support vector machine, a regression tree, an ensemble of regression trees, or some other machine learning model architecture or combination of architectures.

In some examples, the machine learning model of the assessment platform 404 and/or the operation of chemistry analyzer device 402 may be adjusted based on training such that if the outcome of a determined testing response matches the characteristic and/or testing response of the training values, parameters, and/or images, the machine learning model is reinforced and if the outcome of a determined testing response does not match the characteristic of the training values, parameters, and/or images, the machine learning model and/or operation of chemistry analyzer 402 is modified. In some examples, modifying the machine learning model includes increasing or decreasing a weight of a factor within the neural network of the machine learning model. In other examples, modifying the machine learning model includes adding or subtracting rules during the training of the machine learning model.

In a further aspect, these improvements of analyzing values, parameters, and/or images captured by chemistry analyzer device 402 will in turn improve the accuracy and precision of the calibration sequence and operational parameters of chemistry analyzer device 402. Further, once the chemistry analyzer device 402 has been properly calibrated and determined a characteristic of a biological sample in one or more slides, chemistry analyzer device 402 may transmit instructions that cause a computing device (e.g., the computing device 300) to display one or more graphical indications of the identified characteristic and/or one or more images of the biological sample.

In some example embodiments, the biological sample testing may include analyzing of one or more of the following: (i) blood; (ii) urine; (iii) saliva; (iv) fecal matter; (v) secretion; (vi) excretion; (vii) FNA; (viii) lavage fluids; (ix) body cavity fluids; (x) semen; (xi) ear wax; (xii) skin cells; (xiii) biopsied samples, (xiv) exotics; (xv) cultured cells; (xvi) bacteria; (xvii) worms; (xviii) parasites; and (xix) ear mites, among other possibilities. Test may additionally include one or more of the following: blood coagulation test, polymerase chain reaction (PCR) test, and/or immunoassay, among other possibilities. For example, in some example embodiments, these tests may include one or more of the following blood chemistry tests: SDMA, Total T4 (TT4), Bile Acids, C-reactive Protein (CRP), Progesterone, Fructosamine, and/or Phenobarbital (PHBR), among other possibilities. For example, in some example embodiments, these tests may include one or more of the following blood chemistry profile tests that measure one or more of the following: ALB, ALB/GLOB, ALKP, ALT, AMYL, AST, BUN, BUN/CREA, Ca, CHOL, CK, CI, CREA, CRP, FRU, GGT, GLOB, GLU, K, LAC, LDH, LIPA, Mg, Na, NH₃, PHOS, TBIL, TP, TRIG and/or URIC, among other possibilities. Other examples are possible.

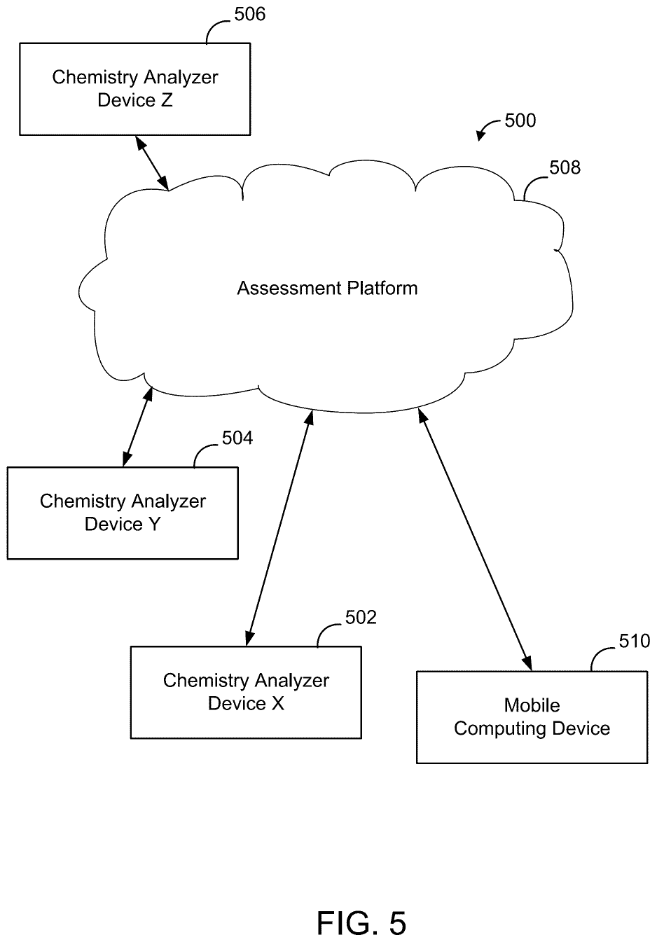

Now referring to FIG. 5, a computing system 500 configured for use with a plurality of chemistry analyzer devices (including chemistry analyzer device X 502, chemistry analyzer device Y 504, and chemistry analyzer device Z 506) and a mobile computing device 510 is illustrated, according to an example embodiment. Like the computing system 400 illustrated in FIG. 4 and described above, all of the components of computing system 500 can be physical systems made up of physical devices, cloud-based systems made up of cloud-based devices that store program logic and/or data of cloud-based applications and/or services (e.g., perform at least one function of a software application or an application platform for computing systems and devices detailed herein), or some combination of the two.

In any event, like computing system 400, computing system 500 can use one or more of chemistry analyzer device X 502, chemistry analyzer device Y 504, and chemistry analyzer device Z 506 to collect data from a number of sources, including from a database of values (e.g., quality control responses and/or concentration values), parameters, and/or images related to the calibration of chemistry analyzer device X 502, chemistry analyzer device Y 504, and chemistry analyzer device Z 506 and/or the testing of biological samples therein. These values (e.g., quality control responses and/or concentration values), parameters, and/or images may be uploaded to an assessment platform 508 and characteristics of these values, parameters, and/or images may be output to a mobile computing device 510.

In some examples, values (e.g., reflectance values), parameters, and/or images that are captured by the one or more of chemistry analyzer device X 502, chemistry analyzer device Y 504, and/or chemistry analyzer device Z 506 can be stored within a memory, such as a memory of chemistry analyzer 100 and/or computing device 300, to be subsequently analyzed.

For example, in some embodiments, one or more of chemistry analyzer device X 502, chemistry analyzer device Y 504, and/or chemistry analyzer device Z 506 may generate, from a control analyte solution having a predetermined concentration, a quality control sample for the control analyte solution. One or more of chemistry analyzer device X 502, chemistry analyzer device Y 504, and/or chemistry analyzer device Z 506 may perform a quality control test on the quality control sample to determine a characteristic of the quality control sample. Based on the quality control test, one or more of chemistry analyzer device X 502, chemistry analyzer device Y 504, and/or chemistry analyzer device Z 506 may generate a quality control response (e.g., indicating whether the characteristic differs from a reference characteristic generated at a reference chemistry analyzer). One or more of chemistry analyzer device X 502, chemistry analyzer device Y 504, and/or chemistry analyzer device Z 506 may store the quality control response and may generate a quality control representation (e.g., a quality control chart) based on the quality control response.

Based on the quality control representation, one or more of chemistry analyzer device X 502, chemistry analyzer device Y 504, and/or chemistry analyzer device Z 506 may determine whether a particular operating parameter (e.g., metering parameter used to generate the quality control sample) is out of the predetermined tolerance and may adjust an internal subsystem to bring the operating parameter within the predetermined tolerance. Additionally or alternatively, in examples, one or more of chemistry analyzer device X 502, chemistry analyzer device Y 504, and/or chemistry analyzer device Z 506 may transmit one or more of these values to assessment platform 508 for further analysis, including for updated values and/or instructions determined for one or more of chemistry analyzer device X 502, chemistry analyzer device Y 504, and/or chemistry analyzer device Z 506 by assessment platform 508 based on the transmitted data.

For example, assessment platform 508 may use a quality control representation (e.g., a quality control chart) determined by a particular chemistry analyzer device (e.g., chemistry analyzer device X 502) to update a quality control representation (e.g., a previously generated quality control chart) stored in the assessment platform and/or one or more other chemistry analyzer devices (e.g., chemistry analyzer device Y 504 and/or chemistry analyzer device Z 506). In this regard, in examples, the analysis and calibration undertaken by a particular chemistry analyzer device may be utilized and leveraged to improve the accuracy, precision, and technical operation of other chemistry analyzer devices, as well as of the assessment platform itself. Other examples are possible.

In a further aspect, in examples, the assessment platform 508 may transmit instructions to the mobile computing device 510 to cause a graphical user interface to display a graphical indication of the identified update, sample characteristic, and/or testing response.

In some examples, the assessment platform 508 may analyze and evaluate one or more calibration parameters and/or testing response by utilizing one or more of: (i) an artificial neural network, (ii) a support vector machine, (iii) a regression tree, or (iv) an ensemble of regression trees, as well as by training one or more machine learning models using data associated values (e.g., reflectance values), parameters, and/or images of one or more slides that share a characteristic with previously captured values, parameters, and/or images of one or more slides. As described in further detail above, the training data may include labeled input values, parameters, and/or images (supervised learning), partially labeled input values, parameters, and/or images (semi-supervised learning), or unlabeled input values, parameters, and/or images (unsupervised learning), as well as reinforcement learning, and the machine learning models described herein may include an artificial neural network, a support vector machine, a regression tree, an ensemble of regression trees, or some other machine learning model architecture or combination of architectures. Other examples are possible.

### Example Methods and Aspects

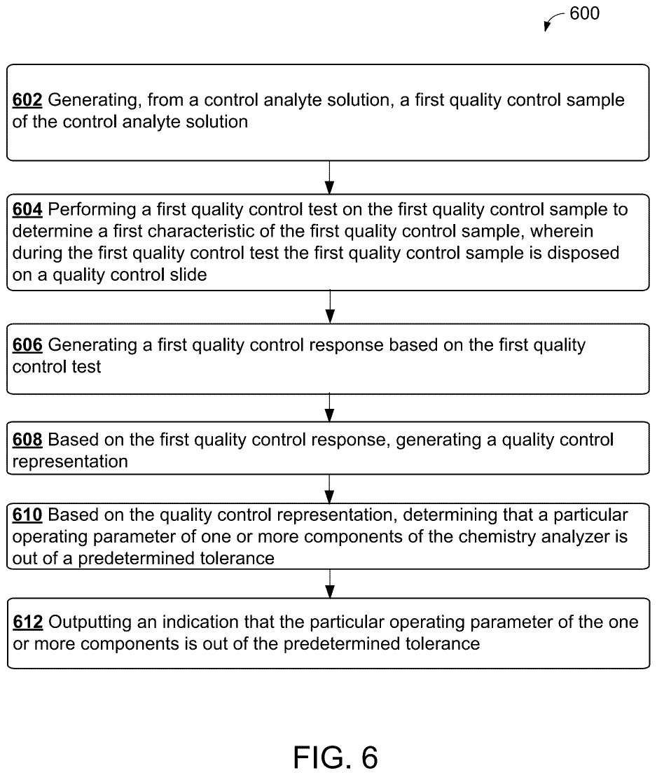

Now referring to FIG. 6, an example method 600 for performing quality control on a chemistry analyzer is disclosed. The method 600 shown in FIG. 6 presents an example of a method that could be used with the components shown in FIGS. 1-5, for example. Further, devices or systems may be used or configured to perform logical functions presented in FIG. 6. In other examples, components of the devices and/or systems may be arranged to be adapted to, capable of, or suited for performing the functions, such as when operated in a specific manner. In examples, the method 600 may include one or more operations, functions, or actions as illustrated by one or more of blocks 602-614. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 602, in examples, the method 600 for performing quality control on the chemistry analyzer includes generating, from a control analyte solution, a first quality control sample of the control analyte solution. In examples, the control analyte solution has a predetermined concentration of a control analyte. In examples, the first quality control sample comprises a first concentration of the control analyte. For example, referring to FIG. 1, the control analyte solution generation unit 130 generate, from the control analyte solutions 126A, 126B having the predetermined concentrations 128A, 128B, respectively, the quality control sample 150A. The quality control sample 150A may be generated to have the target concentration 151A.

At block 604, in examples, the method 600 includes performing a first quality control test on the first quality control sample to determine a first characteristic of the first quality control sample. During the first quality control test the first quality control sample is disposed on a quality control slide. For example, referring to FIG. 1, the quality control testing unit 132 may perform the quality control test 152A on the quality control sample 150A to determine the characteristic 155A of the quality control sample 150A. The quality control test 152A may include applying the quality control sample 150A to one or more quality control slides 120.

At block 606, in examples, the method 600 includes generating a first quality control response based on the first quality control test. For example, referring to FIG. 1, the slide sensor 134 may generate the quality control response 154A based on the quality control test 152A.

At block 608, in examples, the method 600 includes, based on the first quality control response, generating a quality control representation. In example embodiments, the quality control representation may include a quality control chart. For example, referring to FIG. 1, the control representation generation unit 136 may generate a quality control chart 156 based at least on the quality control response 154A.

At block 610, in examples, the method 600 includes, based on the quality control representation, determining that a particular operating parameter of one or more components of the chemistry analyzer is out of a predetermined tolerance. For example, referring to FIG. 1, the operating parameter monitor 138 may determine that the operating parameter 158 is out of the predetermined tolerance based on the quality control chart 156.

At block 612, in examples, the method 600 includes outputting an indication that the particular operating parameter of the one or more components is out of the predetermined tolerance. For example, referring to FIG. 1, the output unit 142 may output an indication that the operating parameter 158 is out of the predetermined tolerance.

In some examples, the method 600 may include adjusting at least one subsystem within the chemistry analyzer to bring the particular operating parameter of the one or more components within the predetermined tolerance. For example, referring to FIG. 1, the subsystem adjustment unit 140 may adjust at least one subsystem within the chemistry analyzer 100 to bring the operating parameter 158 within the predetermined tolerance.

In some examples of the method 600, the first characteristic is measured by one or more sensors of the chemistry analyzer. In some examples of the method 600, the first characteristic comprises one of (i) a reflectance of the first quality control sample or (ii) a reflective density of the first quality control sample. In some examples of the method 600, the first characteristic comprises a concentration of the quality control sample. In some examples of the method 600, the first characteristic comprises a pressure response. In some examples, more than one measurement

In some examples of the method 600, generating the first control response comprises comparing the first characteristic to one or more reference characteristics of a reference quality control sample. In some examples, the reference quality control sample is generated by a reference chemistry analyzer. In some examples, the one or more reference characteristics are received from an evaluation platform.

In some examples, the method 600 includes transmitting the first quality control response to the evaluation platform.

In some examples of the method 600, the one or more components comprises one or more components of an optics module of the chemistry analyzer. In some examples of the method 600, determining that the particular operating parameter of one or more components of the chemistry analyzer is out of the predetermined tolerance comprises determining that one or more components of the optics module of the chemistry analyzer is out of the predetermined tolerance. In some examples, adjusting the at least one subsystem within the chemistry analyzer to bring the particular operating parameter of the one or more components comprises adjusting the one or more components of the optics module within the predetermined tolerance.

In some examples of the method 600, generating the first quality control sample of the control analyte solution comprises performing a cross dilution process using the control analyte solution. In some examples of the method 600, automated metering capabilities of the chemistry analyzer are used to generate the first quality control sample of the control analyte solution.

In some examples, the method 600 includes generating a plurality of quality control samples of the control analyte solution. Each quality control sample of the plurality of quality control samples is generated to have a different concentration, and the first quality control sample is included in the plurality of quality control samples. In examples, the method 600 may also include performing, by the chemistry analyzer, quality control tests on each quality control sample of the plurality of quality control samples. In examples, the method 600 may also include generating, by the chemistry analyzer, quality control responses based on the quality control tests. The quality control representation may be based on the quality control responses.

In some examples, the method 600 includes accessing a control analyte consumable. In examples, the first quality control sample is generated using the control analyte consumable. In some examples, the control analyte consumable comprises a first container holding control analyte solution having a first predetermined concentration of the control analyte, a second container holding control analyte solution having a second predetermined concentration of the control analyte that is less than the first predetermined concentration, and a third container that is empty. In some examples of the method 600, generating the first quality control sample of the control analyte solution comprises mixing, in the third container, the control analyte solution in the first container with the control analyte solution in the second container. In some examples, the first concentration is less than the first predetermined concentration and greater than the second predetermined concentration.

In some examples of the method 600, the control analyte comprises one of alkaline phosphatase, blood urea nitrogen, creatinine, or glucose.

The method 600 of FIG. 6 enables the chemistry analyzer 100 to self-calibrate and prepare control analyte solutions 126 with accurate levels (e.g., concentrations). For example, based on the comparison of the characteristic 155A to reference characteristic 255A from a reference chemistry analyzer, the chemistry analyzer 100 may adjust its automated metering parameters such that the characteristics 155 are within an acceptable margin of error. As a result, when the chemistry analyzer 100 is used by a customer or technician, the calibrated automated metering parameters of the chemistry analyzer may be used to prepare quality control fluids having accurate concentrations for use in analyzing biological samples.

In one aspect, a non-transitory computer-readable medium, having stored thereon program instructions that, when executed by one or more processors, cause a controller of a chemistry analyzer to perform operations, the operations including generating, from a control analyte solution having a predetermined concentration of a control analyte, a first quality control sample of the control analyte solution. The first quality control sample is generated to have a first concentration of the control analyte. In examples, the operations include performing a first quality control test on the first quality control sample to determine a first characteristic of the first quality control sample. The first quality control test includes applying the first quality control sample to a quality control slide. In examples, the operations include generating a first quality control response based on the first quality control test. In examples, the operations include storing the first quality control response in a database. In examples, the operations include generating a quality control representation (e.g., a quality control chart) based at least on the first quality control response. In examples, the operations include determining that a particular operating parameter is out of tolerance based on the quality control representation (e.g., a quality control chart). In examples, the operations include adjusting at least one subsystem within the chemistry analyzer to bring the particular operating parameter within tolerance.

The singular forms of the articles “a,” “an,” and “the” include plural references unless the context clearly indicates otherwise. For example, the term “a compound” or “at least one compound” can include a plurality of compounds, including mixtures thereof.

Various aspects and embodiments have been disclosed herein, but other aspects and embodiments will certainly be apparent to those skilled in the art. Additionally, the various aspects and embodiments disclosed herein are provided for explanatory purposes and are not intended to be limiting, with the true scope being indicated by the following claims.

We claim:

1. A method for performing quality control on a chemistry analyzer, the method comprising:
generating, from a control analyte solution, a first quality control sample of the control analyte solution;
performing a first quality control test on the first quality control sample to determine a first characteristic of the first quality control sample, wherein during the first quality control test the first quality control sample is disposed on a quality control slide;
generating a first control response based on the first quality control test;
based on the first quality control response, generating a quality control representation;
based on the quality control representation, determining that a particular operating parameter of one or more components of the chemistry analyzer is out of a predetermined tolerance; and
outputting an indication that the particular operating parameter of the one or more components is out of the predetermined tolerance.

2. The method of claim 1, further comprising adjusting at least one subsystem within the chemistry analyzer to bring the particular operating parameter of the one or more components within the predetermined tolerance.

3. The method of claim 1, wherein the control analyte comprises one or more of alkaline phosphatase, blood urea nitrogen, creatinine, or glucose.

4. The method of claim 1, wherein generating the first quality control sample comprises performing a cross dilution process using the control analyte solution.

5. The method of claim 1, wherein the first characteristic is measured by one or more sensors of the chemistry analyzer.

6. The method of claim 1, wherein the first characteristic comprises at least one of: (i) a reflectance of the first quality control sample; or (ii) a reflective density of the first quality control sample.

7. The method of claim 1, wherein the first characteristic comprises a concentration of the quality control sample.

8. The method of claim 1, wherein the first characteristic comprises a pressure response.

9. The method of claim 1, wherein generating the first control response comprises comparing the first characteristic to one or more reference characteristics of a reference quality control sample.

10. The method of claim 9, wherein the one or more reference characteristics are received from an evaluation platform.

11. The method of claim 1, wherein the quality control representation comprises a quality control chart.

12. The method of claim 1, wherein the one or more components comprises one or more components of an optics module of the chemistry analyzer.

13. The method of claim 12, wherein determining that the particular operating parameter of one or more components of the chemistry analyzer is out of the predetermined tolerance comprises determining that one or more components of the optics module of the chemistry analyzer is out of the predetermined tolerance; and adjusting the at least one subsystem within the chemistry analyzer to bring the particular operating parameter of the one or more components comprises adjusting the one or more components of the optics module within the predetermined tolerance.

14. The method of claim 1, further comprising:
generating a plurality of quality control samples of the control analyte solution, wherein each quality control sample of the plurality of quality control samples is generated to have a different concentration, and wherein the first quality control sample is included in the plurality of quality control samples;
performing quality control tests on each quality control sample of the plurality of quality control samples; and
generating quality control responses based on the quality control tests, wherein the quality control representation is based on the quality control responses.

15. The method of claim 1, further comprising accessing a control analyte consumable, wherein the first quality control sample is generated using the control analyte consumable.

16. The method of claim 15, wherein the control analyte consumable comprises:
a first container containing control analyte solution having a first predetermined concentration of the control analyte;
a second container containing control analyte solution having a second predetermined concentration of the control analyte that is less than the first predetermined concentration; and
a third container, and wherein generating the first quality control sample of the control analyte solution comprises mixing, in the third container, the control analyte solution in the first container with the control analyte solution in the second container.

17. The method of claim 16, wherein the first concentration is less than the first predetermined concentration and greater than the second predetermined concentration.

18. The method of claim 11, wherein the control analyte comprises one or more of alkaline phosphatase, blood urea nitrogen, creatinine, or glucose.

19. The method of claim 1, wherein the control analyte solution is generated by the chemistry analyzer.

20. The method of claim 1, wherein the first quality control sample is generated by the chemistry analyzer.

21. A chemistry analyzer comprising:
one or more processors; and
a tangible, non-transitory computer-readable medium comprising instructions that, when executed by the one or more processors, cause the one or more processors to perform operations comprising:
generating, from a control analyte solution, a first quality control sample of the control analyte solution;
performing a first quality control test on the first quality control sample to determine a first characteristic of the first quality control sample, wherein during the first quality control test the first quality control sample is disposed on a quality control slide;
generating a first control response based on the first quality control test;
based on the first quality control response, generating a quality control representation;
based on the quality control representation, determining that a particular operating parameter of one or more components of the chemistry analyzer is out of a predetermined tolerance; and
outputting an indication that the particular operating parameter of the one or more components is out of the predetermined tolerance.

22. A tangible, non-transitory computer-readable medium comprising instructions that, when executed by one or more processors, cause a controller of a chemistry analyzer to perform operations comprising:
generating, from a control analyte solution, a first quality control sample of the control analyte solution;
performing a first quality control test on the first quality control sample to determine a first characteristic of the first quality control sample, wherein during the first quality control test the first quality control sample is disposed on a quality control slide;
generating a first control response based on the first quality control test;
based on the first quality control response, generating a quality control representation;
based on the quality control representation, determining that a particular operating parameter of one or more components of the chemistry analyzer is out of a predetermined tolerance; and
outputting an indication that the particular operating parameter of the one or more components is out of the predetermined tolerance.
